(19) United States
(12) Patent Application Publication
(10) Pub. No.: US 2004/0029860 A1
(43) Pub. Date: Feb. 12, 2004

(54) Anti-proliferative drugs

(76) Inventors: Irit Gil-Ad, Herzelia (IL); Abraham Weizman, Tel Aviv (IL)

Correspondence Address: NATH & ASSOCIATES, 1030 15th STREET, 6TH FLOOR, WASHINGTON, DC, 20005, US

(21) Appl. No.: 10/432,875

(22) PCT Filed: Nov. 29, 2001
(86) PCT No.: PCT/IL01/01105

(30) Foreign Application Priority Data
Nov. 29, 2000 (IL) 139975

(51) Int. Cl. A61K 31/553; A61K 31/554; A61K 31/5513; A61K 31/137

(52) U.S. Cl. 514/211.11; 514/220; 514/221; 514/651

(57) ABSTRACT

The present invention relates to methods for the treatment of diseases associated with hyper-proliferation of cells by administering to a subject in need a therapeutically effective amount of at least one psychotropic agent. Specific proliferative diseases against which psychotropic agents were found to be effective are cancer, including multi-drug resistant cancer and diseases associated with hyper-proliferation of the skin cells, such as psoriasis and hyperkeratosis.

### FIELD OF THE INVENTION

The present invention is generally in the field of pharmaceutical compositions and methods for the treatment of disease and disorders, and in particular concerns proliferative diseases such as cancer and various skin disorders.

### BACKGROUND OF THE INVENTION

Psychotropics are drugs used for the therapy of schizophrenia and other psychiatric disorders. There have been several studies indicating their effect in other, unrelated diseases. Silver et al., (Society of Biological Psychiatry, 35:824-826, (1999)) studied the inhibitory effect of several anti-psychotic drugs, including haloperidol and fluphenazine on human neuroblastoma cell lines, and demonstrated that haloperidol, flupentixol, fluphenazine, dopamine and desmethyl imipramine had an inhibitory effects on cell numbers.

Other studies further showed that phenothiazines have anti-proliferative effects on some tumor cells such as leukemic cells, melanoma, glioma and leukemia (Nordenberg et al., Biochemical Pharmacology, 58:1229-1239, (1999)).

In addition there exists at least one publication, U.S. Pat. No. 5,104,858, teaching the sensitizing of multidrug resistant cells to anti-tumor agents by contacting the cells with some phenothiazines and thioxanthenes.

With regard to antidepressants, conflicting reports exist. Clomipramine, imipramine and citalopram were found to induce apoptosis in myeloid leukemia HL-60 cells (Xia et al. J. Biochem Mol Toxicol 13:338-47 (1999)).

The serotonin reuptake inhibitors fluoxetine and zimelidine inhibited proliferation of prostate carcinoma cells, an effect attributed to inhibition of serotonin uptake (Abdul M et al. J Urol 154:247-50 (1995)). Other studies however, showed that in vivo administration of fluoxetine and amitryptiline to mice increased the development of fibrosarcoma, melanoma and breast tumors (Brandes L J, et al. Cancer Res 52:3796-00 (1992)).

Other studies in human showed that antidepressant medications (tricyclic and paroxetine) are associated with elevation of risk in breast cancer (Cotterchio M. et al. Am J Epidemiol, 151:951-7 (2000)), or have no effect on breast cancer at all, as recently reported (Wang P. S. J. Clin. Epidemiol. 54:728-34, (2001)).

Several publications also described the use of psychotropic and neurotropic agents in treating patients with skin diseases having clear discernible psychiatric symptoms, such as psoriasis associated with major depression, vitiligo resulting in social anxiety and delusions of parasitosis (Gupta M. A. et al. J. Am. Dermatol 14(4):633-645 (1986); Tennyson H and Levine N. Dermatol Clin. 19(1):179-197 (2001)). Yet, in another publication Fluoxetine-induced psoriasis was reported (Hemlock C. et al. Ann. Pharmacother. 26(2):211-212 (1992)).

### SUMMARY OF THE INVENTION

The present invention is based on several surprising findings concerning empiric results obtained with several psychotropic drugs.

First, the present invention is based on the finding that clozapine and clotiapine (tricyclic neuroleptic and antipsychotic agents), paroxetine (bicyclic antidepressant) and fluoxetine (monocyclic antidepressant) and other related cyclic psychotropic drugs are effective against numerous tumors, including glioma, melanoma, neuroblastoma, colon, lung and prostate cancers (both hormone dependent and hormone independent) as well as against multi drug resistance (MDR) B16 melanoma cells (known to be resistant to doxorubicin and colchicine), Neuroblastoma (SH-SY5T resistant to 5-FU and doxorubicin).

Thus, according to a first aspect of the invention there is provided a method for the treatment of proliferative diseases comprising administering to a subject in need a therapeutically effective amount of at least one active ingredient, said active ingredient is a cyclic psychotropic agent selected from tricyclic neuroleptic and antipsychotic agents, bicyclic antidepressants and monocyclic antidepressants, with the proviso that said tricyclic neuroleptic and antipsychotic agents are not phenotiazines or thioxantenes and when said active ingredient is a monocyclic antidepressant, said proliferative disease is not prostate cancer.

The term “treatment” as used herein refers to the administering of a therapeutic amount of the psychotropic which is effective to ameliorate undesired symptoms associated with the proliferative disease, effective to prevent the manifestation of such symptoms before they occur, effective to slow down the progression of the proliferative disease (as may be evident from changes in tumor size, formation of metastases etc.), effective to slow down the deterioration of symptoms, effective to enhance the onset of remission period (e.g. in case of psoriasis), effective to slow down the irreversible damage caused in the progressive chronic stage of the disease, effective to delay the onset of said progressive stage, effective to lessen the severity or cure the disease, effective to improve survival rate or more rapid recovery, or effective to prevent the disease form occurring or a combination of two or more of the above.

The term “psychotropic agent” as used herein refers to chemical compounds all of which comprise at least one aromatic ring and are used as CNS active agents. In the following where the name of a specific cyclic psychotropic drug is given it should be understood that this term refers not only to the formula of the drug as given for example in, chemical abstracts or medicinal manuscripts (e.g. in Psychotropics 2000/2001 Lundbck Ed.) but also to small modifications in the formula such as those which increase stability; increase permeability to cells or decrease permeability to the blood brain barrier (BBB) cause slow release and the like, nevertheless, maintain the biological activity of the agent against hyper-proliferative cells.

According to one embodiment of this aspect of the invention, the tricyclic neuroleptic and antipsychotic agent is a derivative of dibenzothiepines, dibenzoazepines, dibenzothiazepines, dibenzodiazepines or dibenzooxazepines and according to one preferred embodiment, the tricyclic neuroleptic and antipsychotic agent is clozapine or clotiapine.

According to yet another embodiment of the invention, the active agent is a bicyclic antidepressant and said bicyclic antidepressant is preferably paroxetine.

Yet further, the active ingredient of the present invention may be a monocyclic antidepressant and according to one embodiment of the invention said monocyclic antidepressant is a phenylpropylamine derivative. Preferred monocyclic antidepressants include phenoxy-3-propylamine derivatives, such as tomoxetine, nisoxetine and most preferably fluoxetine.

By one embodiment, the proliferative diseases are tumors including both benign as well as malignant tumors. In particular, the tumors treated by the cyclic psychotropic agents defined above are glioma, melanoma, neuroblastoma, colon, lungs, breast and prostate cancer, multi-drug resistant cancers as well as cancers involved with mutated p53 gene.

As may be appreciated by those of skill in the art, at times, it would be preferable to administer the active psychotropic agent in combination with cytotoxic drugs, such as doxorubicin. Thus, the present invention also relates to treatment of proliferative diseases in which the psychotropic agent is administered in combination with one or more cytotoxic drug.

By the term “combination” used herein it should be understood that the cytotoxic drug may be provided to the subject in need, either prior to administration of the psychotropic agent, concomitant with administration of said psychotropic agent (co-administration) or shortly thereafter. Thus, the invention should be understood as relating to any form of combination between the active psychotropic agent and a cytotoxic drug.

Another finding on which the present invention is based is that psychotropic agents, such as clozapine (tricyclic neuroleptic and antipsychotic), clomipramine (tricyclic antidepressant), paroxetine (bicyclic antidepressant) and fluoxetine (monocyclic antidepressant) were effective in sensitizing cancer cells to doxorubicin.

Thus, according to a second of its aspects, the present invention provides a method for sensitizing proliferative cells to a cytotoxic drug, the method comprising administering to a subject in need an amount of said cytotoxic drug in combination with a sensitizing amount at least one psychotropic agent with the proviso that said psychotropic agent is not a phenothiazine or a thioxantene.

The term “sensitizing amount” as used herein refers to any amount of the psychotropic agent which is effective in inducing the toxicity of a drug towards target cells, the drug being at concentrations which without the psychotopic agent would not be toxic to said target cells.

According to one embodiment of this aspect of the invention, the psychotropic agent is a cyclic neuroleptic and antipsychotic agent or a cyclic antidepressant. For example, tricyclic neuroleptic and antipsychotic agents include clozapine while tricyclic antidepressants include clomipramine, amitriptyline, doxepin and imipramine.

Alternatively, according to this aspect of the invention, the active psychotropic agent may be a bicyclic antidepressant, such as paroxetine or a monocyclic antidepressant, such as fluoxetine.

The sensitizing method of the present invention may be applied to any type of cancer cells, including MDR cancer cells. In fact, according to one embodiment of the invention, there is provided a method for sensitizing MDR cancer cells to doxorubicin comprising administering to a subject in need an amount of doxorubicin in combination with a sensitizing amount of at least one psychotropic agent selected from clozapine, clomipramine, fluoxetine and paroxetine.

The present invention is further based on the finding that thioridazine, a member of the phenothiazine family of neurotropic and antipsychotic agents, is effective in sensitizing MDR cancer cells to cytotoxic drugs. While some publications describe phenothiazine-induced cytotoxity, the potentiating effect of thioridazine is only now disclosed.

Thus, the present invention provides a method for sensitizing MDR cancer to a cytotoxic drug comprising administering to a subject in need an amount of said cytotoxic drug in combination with a sensitizing amount of thioridazine.

Another finding on which the present invention is based is that cyclic psychotropics are active against proliferative skin disorders such as psoriasis and hyperkeratosis, and possibly also against basal cell carcinoma.

Thus, according to yet another aspect of the invention there is provided a method for the treatment of proliferative skin disorders which are not associated with psychiatric symptoms comprising administering to a subject in need a therapeutically (e.g. dermatologically) effective amount of at least one psychotropic agent.

By the phrase “which are not associated with psychiatric symptoms” it is meant that the subject in need of the treatment of the present invention does not suffer, in addition to the skin disorder, from any psychiatric disorder which may, directly or indirectly, be the cause or be associated with the formation with the proliferative skin disorder. As mentioned hereinbefore, there are several indications that psychotropic drugs may affect skin disorders which are associated with mental disorders, such as psoriasis associated with major depression, vitiligo resulting in social anxiety and delusions of parasitosis. Notwithstanding these facts, the present invention has now surprisingly shown that irrespective of whether the subject in need suffers from other disorders/diseases, the proliferative the skin disorders may be effectively treated by administration to the said subject a psychotropic agent as defined.

The proliferative skin disorders may include any skin disorder associated with excessive proliferation of the skin cells and include, inter alia, psoriasis, hyperkeratosis and basal cell carcinoma.

According to one embodiment of this aspect of the invention, the psychotropic agent used for treatment of proliferative skin disorders is a phenothiazine, including, preferably, thioridazine, perphenazine and fluphenazine.

However, according to another embodiment of this aspect of the invention, the psychotropic agent is a cyclic antidepressant. Cyclic antidepressants may include tricyclic antidepressants, such as clomipramine, amitriptyline, doxepin and imipramine, bicyclic antidepressants, such as paroxetine, and monocyclic antidepressant, such as fluoxetine.

Administration of the active ingredient according to the present invention may be carried out by any method known in the art for administration of pharmaceuticals. For tumor treatment or for sensitizing tumor cells to a cytotoxic drug the administration in particular includes oral administration, parenteral administration (such as i.v., i.p., s.c.), direct injection to tumor site, as well as administration of slow release substances. The psychotropic agents, both of the cyclic neuroleptics and antipsychotic agents and antidepressants can be administered both during the active chemotherapy stage, optionally together with other known anti-tumor agents having an anti-proliferative activity, as well as for secondary prevention purposes (chronic intake) during remission states. The agents may be especially useful for treatment of tumors which are resistant to doxorubicin and other cytokines, since they are capable of effecting even those tumors which are drug resistant.

For treatment of proliferative skin diseases, oral and parenteral administrations are applicable, however, topical administration is preferable and the active ingredient may be administered as a salve, lotion, ointment (hydrophilic or lipophilic) or suspensions. Acquosum ointments are especially preferred.

The present invention also concerns a pharmaceutical composition for the treatment of proliferative diseases comprising a therapeutically effective amount of at least one active ingredient and a pharmaceutically acceptable carrier, said active ingredient is a cyclic psychotropic agent selected from tricyclic neuroleptic and antipsychotics, bicyclic antidepressants and monocyclic antidepressants as defined hereinabove in connection with the method of treatment of proliferative diseases.

The pharmaceutically acceptable carriers described herein, for example, vehicles, adjuvants, excipients, or diluents, are well-known to those who are skilled in the art and are readily available to the public. It is preferred that the pharmaceutically acceptable carrier be one which is chemically inert to the active compounds and one which has no detrimental side effects or toxicity under the conditions of use. One example of a carrier suitable for topical administration is a standard (aqueosum) eucerinun preparation commonly used by pharmacists.

The choice of carrier will be determined in part by the particular active agent, as well as by the particular method used to administer the composition of the invention. Accordingly, there is a wide variety of suitable formulations of the pharmaceutical composition of the present invention.

The present invention also concerns a pharmaceutical composition for sensitizing proliferative cells to a cytotoxic drug comprising an amount of said cytotoxic drug and a sensitizing effective amount of a psychotropic agent as defined above in connection with the method of the invention for sensitizing proliferative cells to a cytotoxic drug.

According to one preferred embodiment, the present invention provides a pharmaceutical composition for sensitizing MDR cancer cells to doxorubicin comprising an amount of doxorubicin and a sensitizing effective amount of at least one psychotropic agent selected from clozapine, clomipramine, fluoxetine and paroxetine.

According to another aspect, a composition for sensitizing MDR cancer cells to a cytotoxic drug comprising an amount of said cytotoxic drug and a sensitizing effective amount of thioridazine is provided.

The present invention also concerns pharmaceutical compositions for the treatment of proliferative skin disorders which are not associated with psychiatric symptoms comprising a therapeutically effective amount of a psychotropic agent, as defined hereinabove with connection to the method of the invention for the treatment of proliferative skin disorders.

Finally, the invention also concerns the different uses of psychotropic agents against proliferative diseases. For example, the invention concerns the use of a cyclic psychotropic agent selected from tricyclic neuroleptic and antipsychotics, bicyclic antidepressants and monocyclic antidepressants for the preparation of a pharmaceutical composition for the treatment of proliferative diseases, the psychotropic agents being as defined above in connection with the method of the invention for treatment of proliferative diseases.

Further, the use of psychotropic agents for the preparation of a pharmaceutical composition for sensitizing proliferative cells, e.g. MDR cancer cells, to a cytotoxic drug is also disclosed by the present invention with the proviso that said psychotropic agent is not a phenothiazine or a thioxantene, with the exception of thioridazine as the psychotropic agent.

Yet, the invention also concerns the use of psychotropic agents for the preparation of a pharmaceutical composition for the treatment of proliferative skin disorders which are not associated with psychiatric symptoms.

The invention will now be described by way of examples with reference to the accompanying Figures. While the foregoing description describes in detail only a few specific embodiments of the invention, it will be understood by those skilled in the art that the invention is not limited thereto and that other psychotropic agents may be applied to other types of proliferative diseases, without departing from the scope of the invention as defined by the appended claims.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows the effect of paroxetine on viability of primary mouse brain cells, primary neurons and on neuroblastoma cells;

FIGS. 2A-2B shows the effect of trifluoperazine and Fluopentixol; on cell viability of prostate cancer (LN-Cap (FIG. 2A), PC-3 (FIG. 2B));

FIGS. 3A-3C shows the effect of clozapine on viability of prostate LN-Cap cells, melanoma B16 and C6 glioma cells (FIG. 3A); of clotiapine on viability of mouse lung carcinoma cells (FIG. 3B) and of clozapine on mouse melanoma (B16) wild type and MDR cells (FIG. 3C);

FIGS. 4A-4D shows the effect of different antidepressant agents on neuroblastoma SH-SY5T cells (FIG. 4A), 3LL lung carcinoma (FIG. 4B), LN-Cap prostate cells (FIG. 4C) and B16 melanoma cells (FIG. 4D);

FIG. 5 shows the effect of different psychotropic agents on lungs weight in mice 30 days post inoculation with 3LL lung carcinoma cells;

FIGS. 6A-6F shows the effect of cyclic psychotropic on the toxicity of doxorubicin in cancer cell lines. FIG. 6A shows clozapine-induced toxicity in LN-CapAp prostate cells; FIG. 6B shows clomipramine-induced toxicity in B16 melanoma cells, FIG. 6C shows clomipramine-induced toxicity in B16-MDR melanoma cells; FIG. 6D shows paroxetine-induced toxicity in B16 melanoma cells; FIG. 6E shows clompramine-induced toxicity in neuroblastoma SH-SY5T cells; and FIG. 6F shows fluoxetine-induced toxicity in neuroblastoma SH-SY5T cells; FIG. 6G shows clozapine-induced toxicity in glioma C6 cell line.

FIG. 7 shows the effect of perphenazine on apoptosis of neuroblastoma cell-line 24 or 48 hours post administration;

FIG. 8 shows western blot analysis of p53 gene product in glioma C6 cell being induced by thioridazine, clozapine and perprenazine;

FIG. 9 shows the effect of different antidepressant agents on B-16 MDR melanoma cells;

FIG. 10 shows lungs weight of mice inoculated with melanoma B16 cells and treated with thioridazine 21 and 27 days after inoculation;

FIG. 11 shows lung weight of mice inoculated with melanoma cells and treated with thioridazine in drinking water;

FIG. 12 shows lungs weight of mice inoculated with melanoma cells and treated with doxorubicin and doxorubicin+thioridazine (in drinking water);

FIGS. 13A-13B shows the effect of tricyclic psychotropic agents on viability of keratinocytes cells, including HaCat cells (FIG. 13A) and HaCat I5 cells;

FIGS. 14A-14B shows the effect of monocyclic, bicyclic and tricyclic psychotropic agents on viability of HaCat I5 keratinocytes cells (FIG. 14A) or HaCat II4 cell keratinocytes cells (FIG. 14B);

FIGS. 15A-15B shows the effect of phenothiazines; doxorubicin and 5-FU is on HaCat (FIG. 15A) and HaCat I5 (FIG. 15B) keratinocytes cell viability;

FIG. 16 shows the effect of thioridazine on DNA fragmentation in HaCat cells.

### DETAILED DESCRIPTION OF THE INVENTION

### EXAMPLE 1

### Therapeutic Potency of Psychotropic Drugs

Some phenothiazines, tricyclic neuroleptics, and antidepressants, bicyclic antidepressants, monocyclic antidepressants, haloperidol (a butyrophenone) and others were administered to different cell lines. The cytotoxic activities were determined using the neutral red (NR) Almar Blue (AB) and Hoechst dye fluorimetric methods for evaluating DNA content in different cell lines: human neuroblastoma (SK-N-SH) and (SH-SY5T); rat glioblastoma (C6); mouse melanoma (B-16), human prostate (LN-CapAp); and PC-3, mouse lung sarcoma (3LL), and human breast (MCF7). The potential therapeutic potency was calculated as the ratio between the safety of administered daily dose and the mean IC50 (in μM) for each agent. The results are shown hereinbelow in Tables 1A, 1B and 1C.

The results demonstrate the effectiveness of psychotropic drugs in inhibiting proliferation of cancer cells. A particular effect was observed with the cyclic psychotropic drugs clomipramine (tricyclic neuroleptic and anti-depressant), paroxetine (bicyclic neuroleptic and anti-depressant) and fluoxetine (monocyclic neuroleptic and anti-depressant) (Table 1B), while no substantial effect was observed with other groups of the psychotropic agents, such as those exemplified in Table 1C.

The effects of different agents, such as perphenazine, haloperidol, clozapine, risperidone and sulpiride, was also tested in primary tissue cultures including, mouse embryo whole brain culture, mouse-selected embryo, neuronal culture and rat new born myocytic culture. The results obtained (data not shown) demonstrated a marked decrease in responsiveness, or total lack of sensitivity of the whole brain tissue to all agent tested IC₅₀>200 μM (except for perphenazine). The sensitivity of neuronal and myocytic culture showed IC₅₀ for perphenazine of 60 μM and 35-55 μM respectively.

TABLE 1A
Effect of Phenothiazines and Thioxantenes on Viability (Ic50) in Different Cell-Lines

| | | | | | | | | | | Therapeutic index |
|---|---|---|---|---|---|---|---|---|---|---|
| | Neuroblastoma | Neuroblastoma | Glioma | Melanoma | Prostate | Prostate | Breast | Colon | | Mean safe |
| | SK-N-SH | SH-SY5T | C6 | B16 | LN-Cap | PC3 | MCF7 | Ht-29 | Mean ± SD | dose/IC₅₀ |
| Thioridazine | 15 | 11 | 13 | 16 | 14 | 24 | | 19 | 16.5 ± 4.1 | 400 mg/15.5 = 24.2 |
| Chlorpromazine | 100 | 15 | 22 | 46 | | | | | 45.7 ± 33.3 | 600 mg/45.7 = 13.1 |
| Trifluoperazine | | | 14 | 16 | 18 | 19 | | | 16.7 ± 1.9 | 40 mg/16.7 = 2.39 |
| Flupentixiol | | | 14 | 18 | 23 | 18 | | | 18.25 ± 3.2 | 40 mg/18.25 = 2.19 |
| Fluphenazine | 21 | 15 | 20 | 18 | | | | | 18.5 ± 2.2 | 40 mg/18.5 = 2.16 |
| Perphenazine | 21 | 15 | 22 | 23 | 18 | | 21 | 22 | 20.0 ± 2.7 | 40 mg/20.0 = 2.0 |

TABLE 1B
Effect of Other Cyclic Psychotropic Drugs on Cell Viability (IC50) in Different Cell-Lines^{a}

| | | | | | |
|---|---|---|---|---|---|
| Lungs Sarcoma | Neuroblastoma | Melanoma | Melanoma B16 | Prostate | |
| 3LL | SH-SY5T | 16B | MDR | LN-Cap | Mean +/− SD |

Trycylic

| | | | | | | |
|---|---|---|---|---|---|---|
| Imipramine | 12 | 69 | 34 | 39 | | 38.5 +/− 23.5 |
| Clomipramine | <10 | 21 | 20 | 36 | 22 | 19.8 +/− 9.6 |
| Amitriptiline | | | 64 | | | 64 |
| Doxepin | | | 76 | | | 76 |
| Clozapine | 33 | 74 | 32 | 35 | 42 | 36.0 +/− 23 |
| Clotiapine | 40 | | 45 | | | |
| Bicyclic | | | | | | |
| Paroxetine | <10 | 15 | 12 | 11 | 16 | 12.4 +/− 3.2 |
| Monocyclic | | | | | | |
| Fluoxetine | <10 | 30 | 14 | 14 | 17 | 14.1 +/− 9.7 |

^{a}IC50 values expressed in μM

TABLE 1C
Effect Of Cyclic Psychotropic Drugs On Viability (Ic50) In Different Cell-Lines

| | Neuroblastoma | Neuroblastoma | | | |
|---|---|---|---|---|---|
| | SK-N-SH | SH-SY5T | Glioma C6 | Melanoma B16 | Prostate LN-CapAp |
| Sulpiride | Non Toxic | | Non Toxic | | Non Toxic |
| Risperidone | Non Toxic | Non Toxic | | Non Toxic | |
| Mianserin | | Non Toxic | | | Non Toxic |
| Haloperidol | 100 | 1 > 100 | 100 | | |

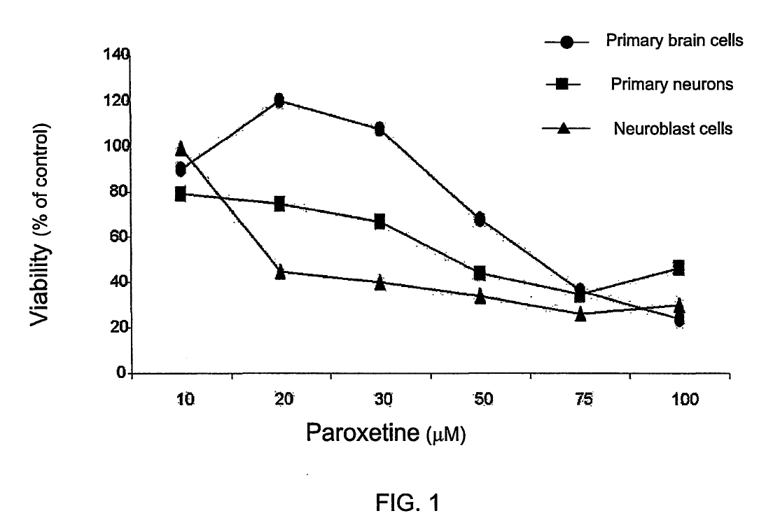

The effect of paroxetine on primary tissue cultures was also evaluated. FIG. 1 shows a comparison between the sensitivity of primary mouse brain, selected neurons (obtained by treatment of mouse whole brain embryo culture with 5 Fluorouridine) and human neuroblastoma cells (SH-SY5T) to paroxetine at concentrations of between 10 μM to 100 μM. The viability of the cells after treatment with paroxetine was determined by neutral red (NR) technology 24 hr. post treatment. As shown in FIG. 1, an increased sensitivity to paroxetine was observed with neuroblastoma cells, as compared to primary tissue.

These results taken together with the results shown in Tables 1A-LC teach that the sensitivity of tumor tissue, such as melanoma, neuroblastoma, prostate and lung carcinoma, to the cytotoxic effect of the tested drugs is significantly higher than that of primary tissue.

The results in Table 1B are supported by the various examples presented hereinafter which show the efficiency of cyclic psychotropic drugs on various cell lines (prostate, melanoma and glioma). As shown in Table 1B, paroxetine (a bicyclic antidepressant) and fluoxetine (a monocyclic antidepressant) were found to be very effective against these cell lines, with an effect similar to that obtained with the highly effective tricyclic antidepressant, clomipramine.

### EXAMPLE 2

### Effect of Phenothiazines (Tricyclic Neuroleptic and Antipsychotic Agents) on Prostate Cells

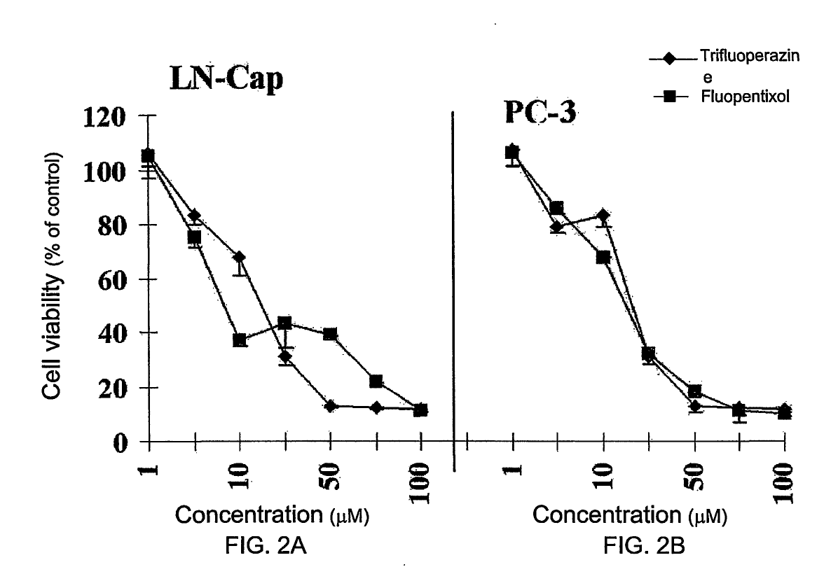

The effect of trifluoperazine and flupentixol was studied in two cell-lines of human prostate cancer: androgen dependent (LN-Cap) and androgen independent (PC-3). FIGS. 2A and 2B show the results of incubation of these cell lines with the agents (at concentrations between 1 μM-100 μM), which indicate that both agents were effective in inhibition of cell proliferation.

### EXAMPLE 3

### Effect of Tricyclic Neuroleptic Drug on Various Malignant Cell Lines

Clozapine at varying concentrations (10 μM-100 μM) was applied to different cancer cell lines, including prostate (LN-CapAp), melanoma B16, C6 glioma, and cell viability was determined as described above.

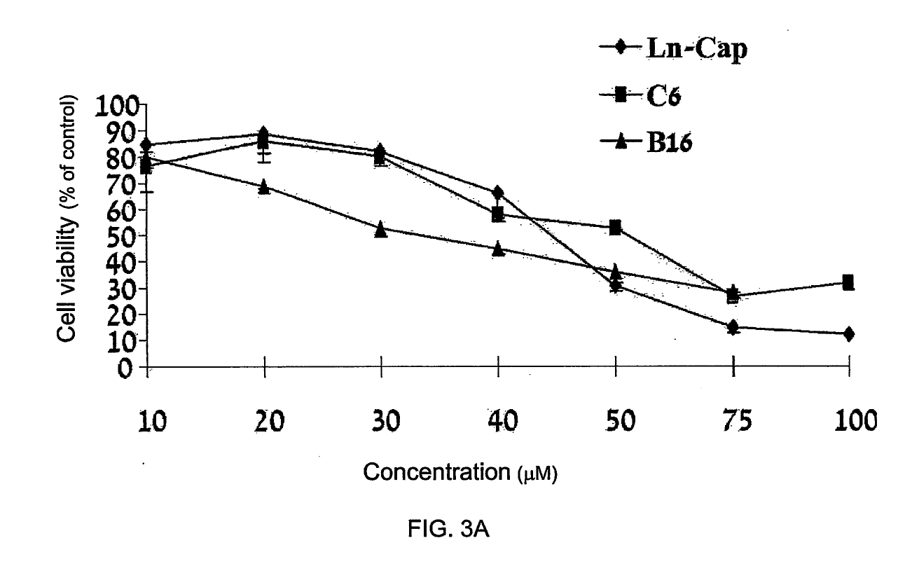

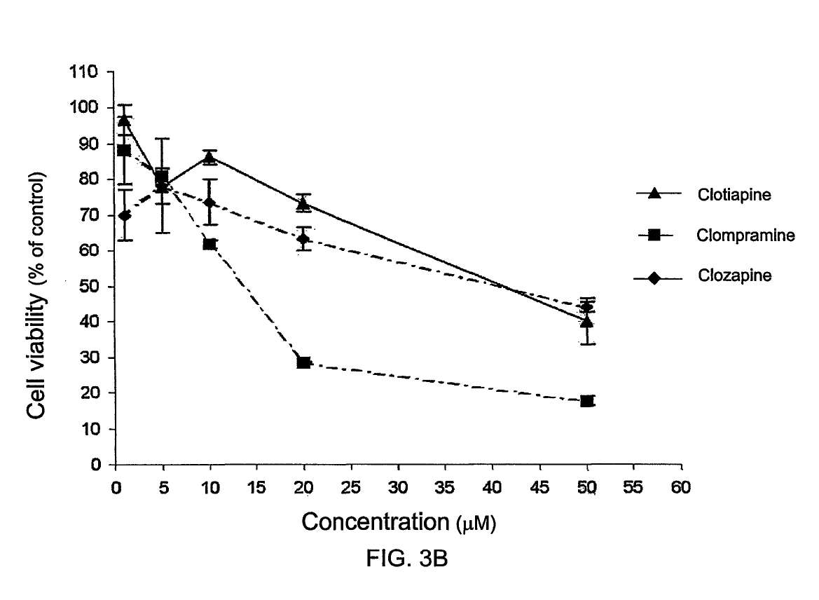

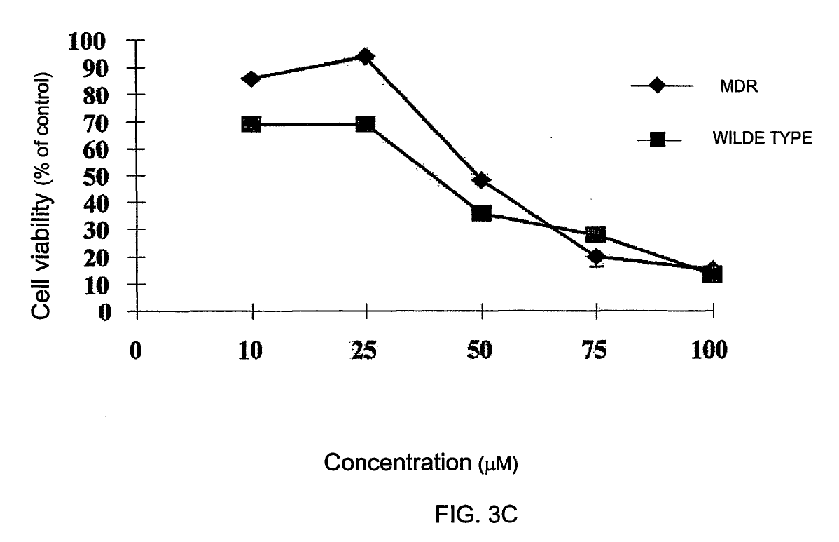

The results are presented in FIG. 3A which shows that in the presence of clozapine, the viability of the tested cell lines was significantly reduced.

In another experiment the effect of clotiapine at the same concentrations on the viability of mouse lung carcinoma (3LL) cells was determined. FIG. 3B which also presents the effect of other tricyclic psychotropic agents on this cell line, teach that also clotiapine is effective in reducing viability of the malignant cells.

In yet another experiment, clozapine at concentrations of between 10 μM-100 μM was applied to mouse melanoma (B16) wild type and MDR cells. The results of this experiment are presented in FIG. 3C which teach that clozapine was effective in reducing viability of both wild type and MDR cancer cells.

### EXAMPLE 4

### Effect of Cyclic Antidepressants on Various Malignant Cell Lines

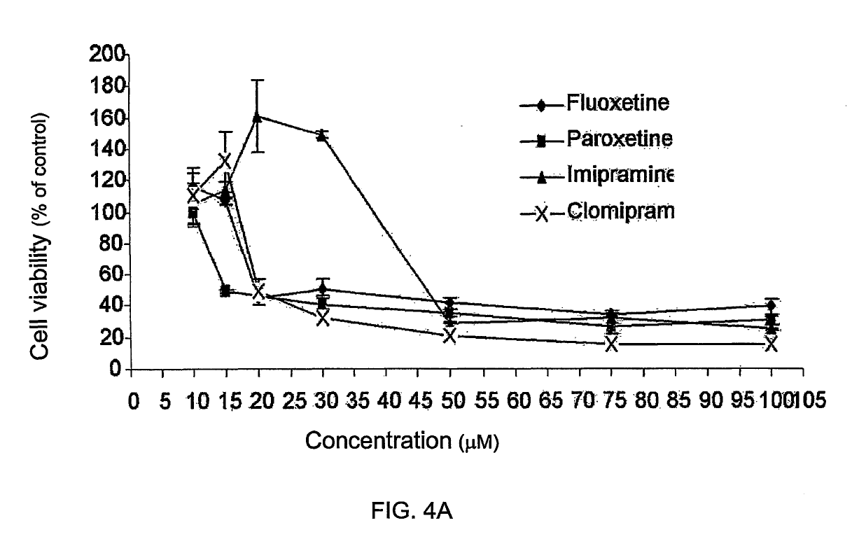

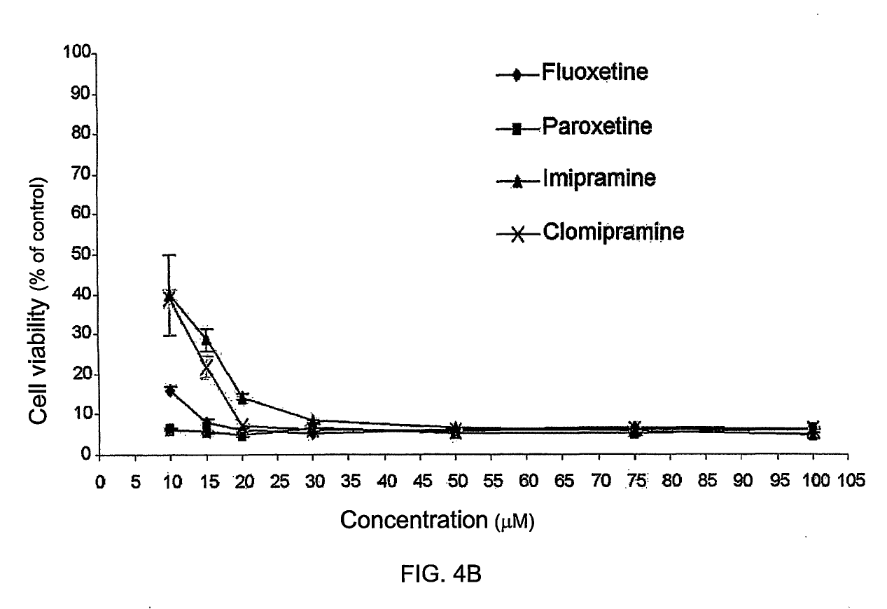

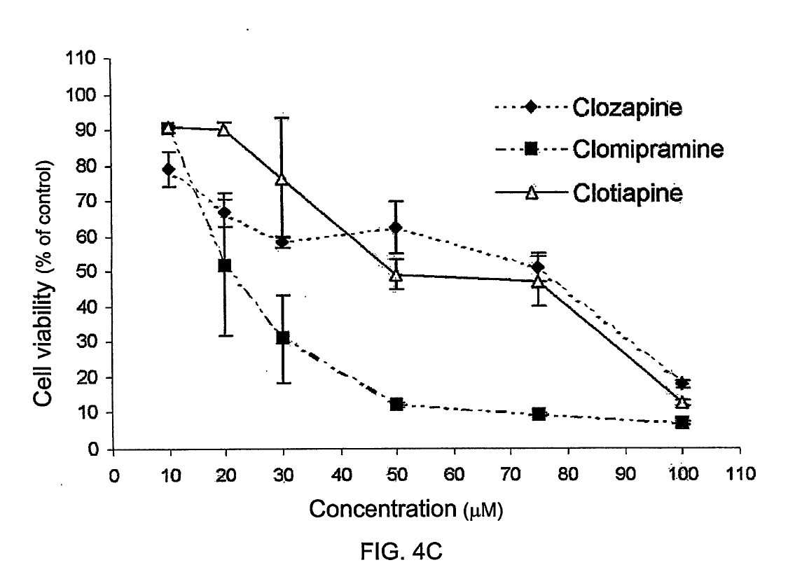

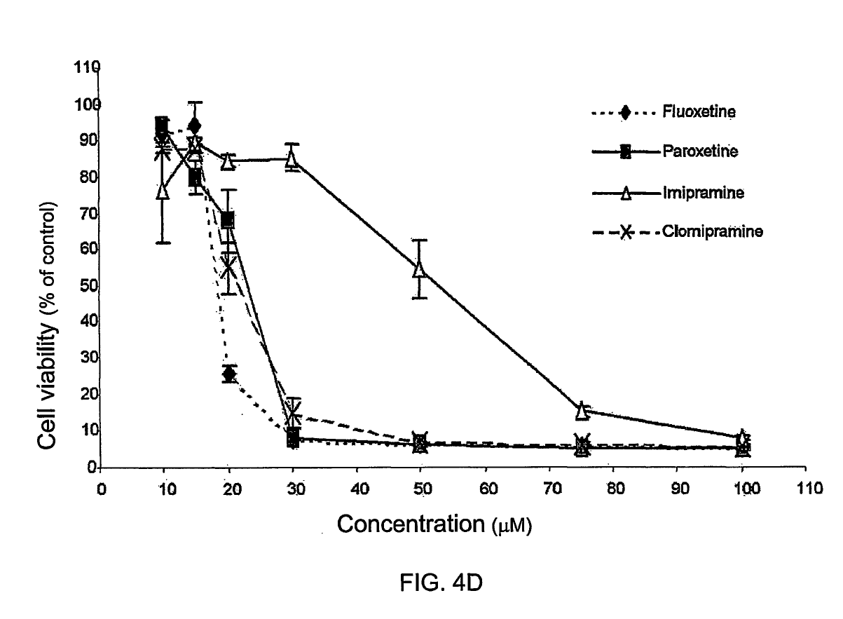

Cyclic antidepressants at varying concentrations (between 10PM and 100 μM) was applied to neuroblastoma SH-SY5T, 3LL lung carcinoma, prostate (LN-CapAp), melanoma B16, and cell viability was determined as described above. Clomipramine (tricyclic), imipramine (tricyclic), paroxetine (bicyclic) and fluoxetine (monocyclic). FIGS. 4A-4E show the effect of the indicated drugs on the difference cell lines, all showing a significant ability to inhibit survival of the different tumors.

### EXAMPLE 5

### In Vivo Effect of Cyclic Psychotropic Agents on Tumors

Female C57 black mice aged 4 weeks were used in order to evaluate the in vivo effect of cyclic psychotropic agents on tumor cells. In particular, animals were divided into 5 groups (6-8 mice each). Animals were inoculated with mouse Lewis lung carcinoma (3LL, 0.5 million each) by i.v. injection to the tail vein and then treated by i.p. injection daily for 3 weeks with the following different agents

(1) saline (control);

(2) Thioridazine (15 mg/kg);

(3) Clomipramine (30 mg/kg);

(4) Fluoxetine (30 mg/kg); and

(5) Paroxetine (30 mg/kg).

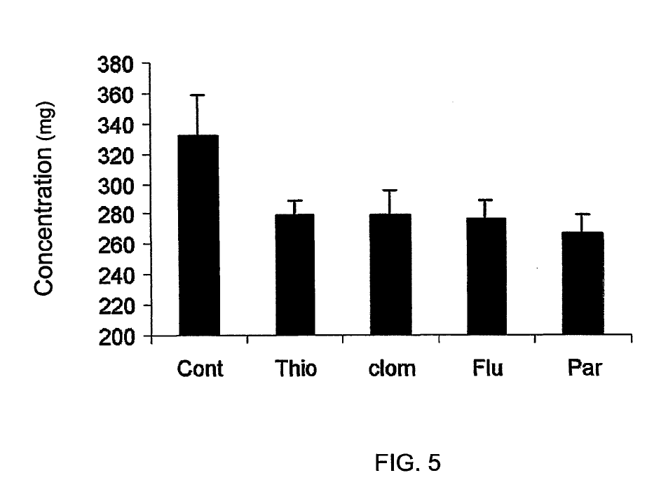

During the treatment period animals were inspected daily and their body weight registered twice a week. The survival rate during four weeks of treatment was: Controls:8/8, thioridazine 7/7, clomipramine 7/7 fluoxetine 5/7 (two animals died on third and forth week, but no signs of lung metastases or other tumor was found), and paroxetine 5/6 (one animal died on third week, with no signs of metastases or tumor). After the four weeks of treatment the mice were sacrificed and their lungs dissected and weighted. Lungs weight is shown in FIG. 5 which teaches that all active agents were effective in reducing tumor size as compared to the control group.

### EXAMPLE 6

### Sensitization of Doxorubicin Cytotoxicity by Low Concentrators of Neuroleptic and Antidepressant Agents

### EXAMPLE 6A

### Clozapine-Induced Toxicity of Doxorubicine in Prostate LN-Cap Cells

Prostate LN-Cap cells were divided into three groups:

1. Cells treated with clozapine alone (20 μM and 25%.

2. Cells treated with doxorubicin alone (1 μM)

3. Cells treated with clozapine and doxorubicin (administered simultaneously) at the concentrations indicated for groups 1 and 2.

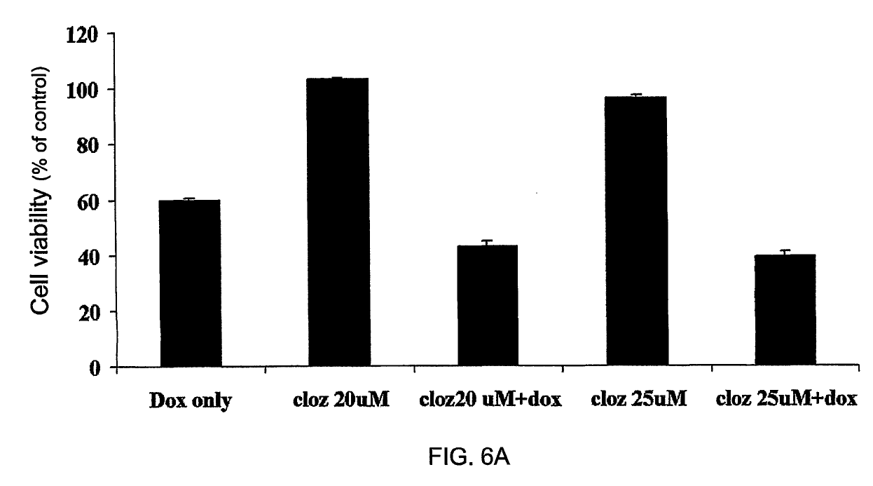

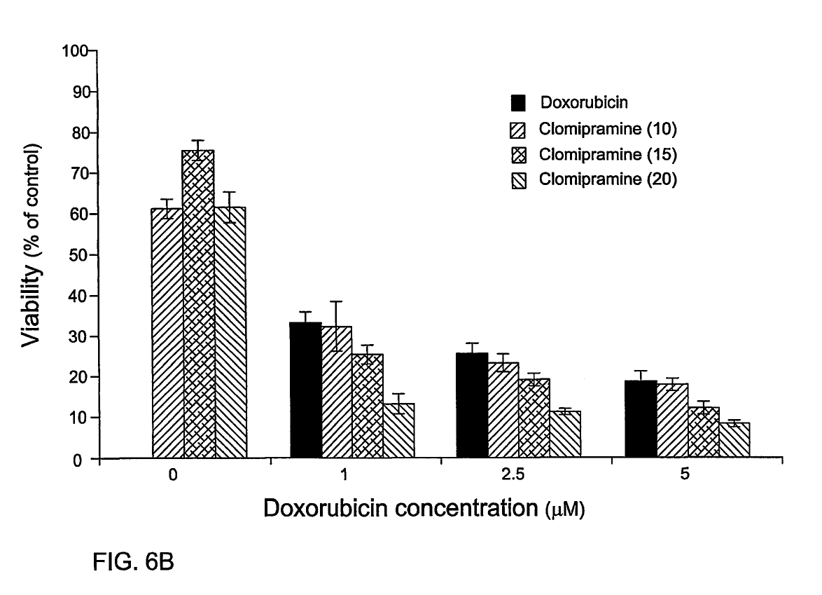

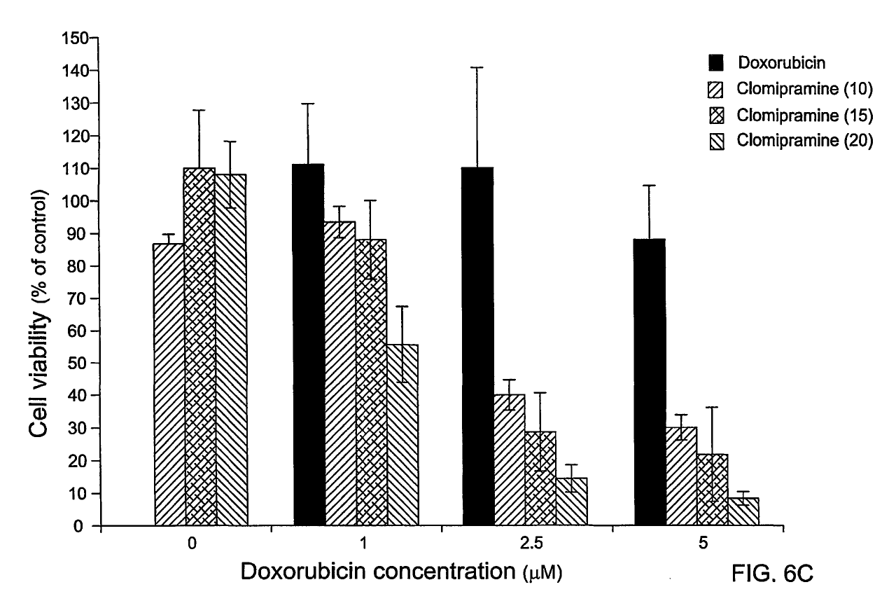

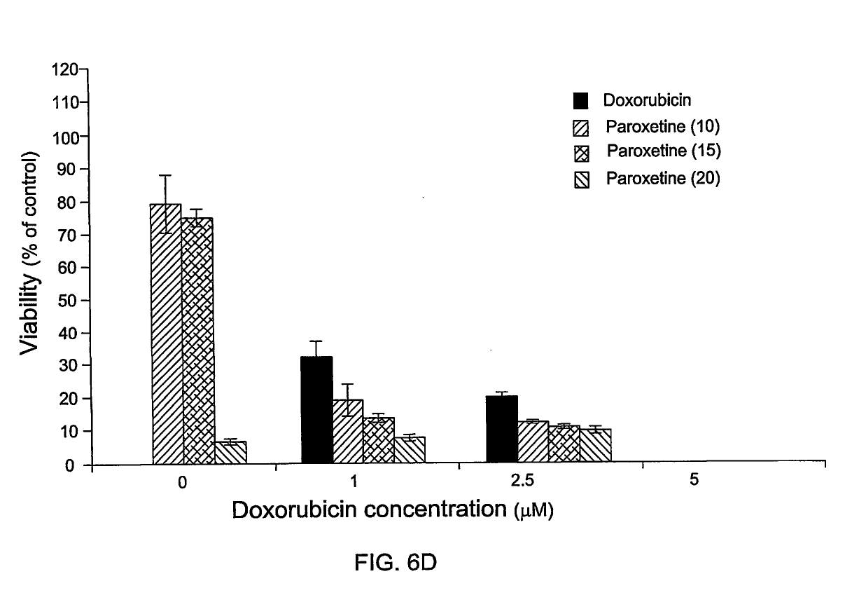

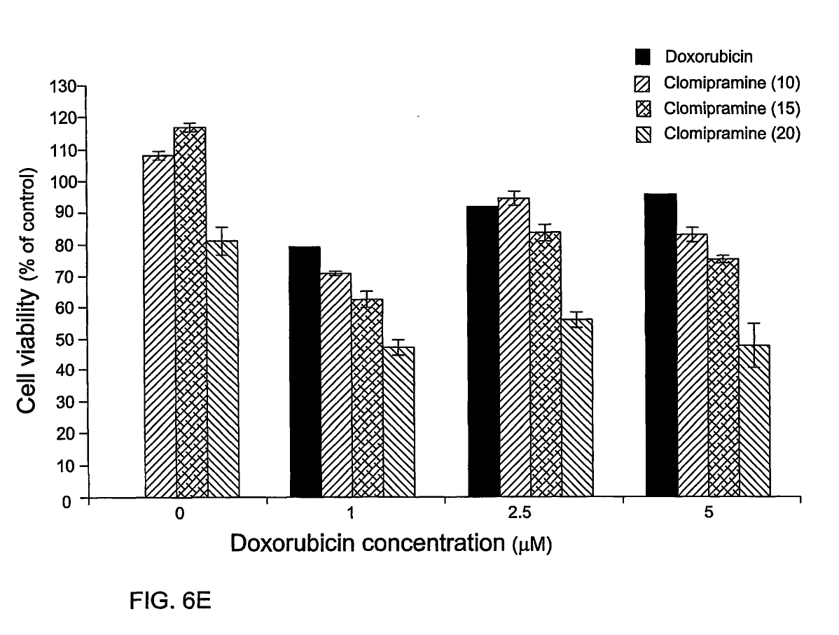

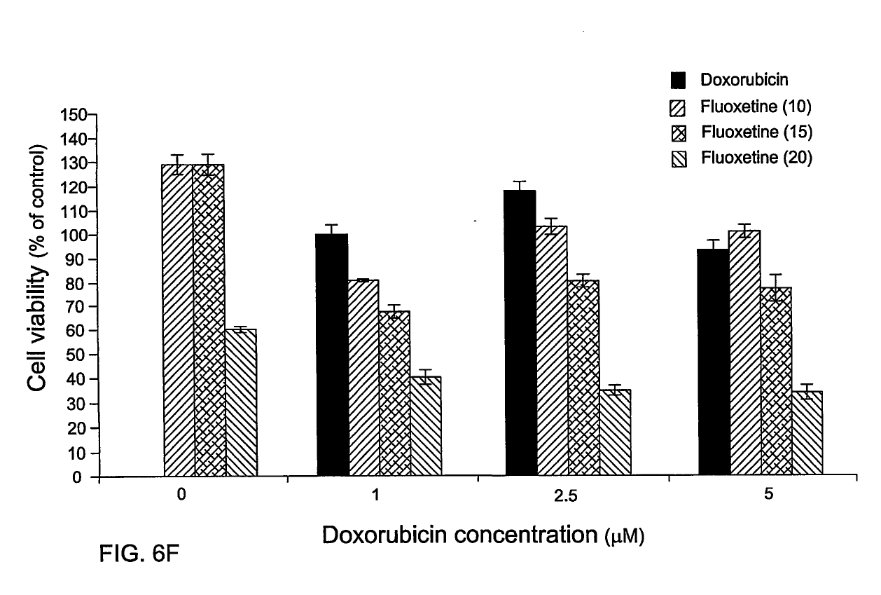

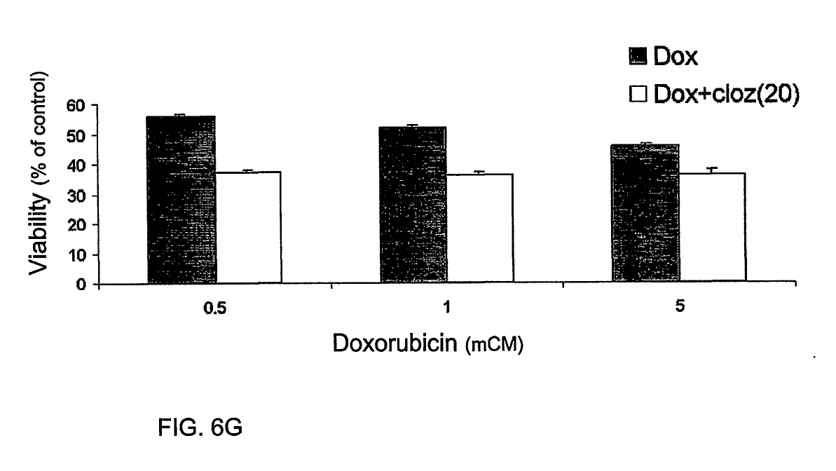

FIG. 6A presents the induction of doxorubicin (a cytotoxic drug widely used in the therapy of malignant diseases) toxicity by clozapine in the prostate cancer cell-line. In particular, the results presented in this Figure demonstrate that at concentrations in which clozapine alone is not effective, its combination with doxorubicine potentiated the toxicity of the latter towards the tested cell line.

### EXAMPLE 6B

### Clomipramine-Induced Toxicity of Doxorubicine in B16 Melanoma Cell Line

The effect of clomipramine, a tricyclic antidepressant on the toxicity of doxorubicin in melanoma B16 was evaluated.

Clomipramine (10, 15, and 20 μM) was applied to B16 melanoma cells either alone or in combination with doxorubicin (1, 2.5 and 5 μM). The results presented in FIG. 6B shows that while low concentrations of clomipramine were effective in reducing cell viability, when administered in combination with the toxic agent, doxorubicin, cell viability was substantially reduced to less than 15% from control.

When clomipramine was applied to MDR B16 melanoma in combination with doxorubicin, cell viability was significantly decreased, in a dose dependant manner, demonstrating the potentiation of doxorubicin by the antidepressant (FIG. 6C). The same effect was also observed with paroxetine when applied to B16 melanoma cells (FIG. 6D).

When applied to doxorubicin resistant neuroblastoma cells (SH-SY5T), clomipramine (FIG. 6E) and fluoxetine (FIG. 6F) where also shown to potentiate the toxicity of doxorubicin, in a dose dependant manner. Finally, when clozapine was applied to glioma C6 cells, it was also shown to induce toxicity of doxorubicin to these cells (FIG. 6G).

### EXAMPLE 7

### Effect of Perphenazine on Apoptosis in Neuroblastoma Cell Lines

Neuroblastoma cell line (SK-NSH) were administered with varying concentrations of perphenazine (2.5-40 μM). The percentage of apoptosis of the cells was determined by flow cytometry of propidium iodide stained cells using a fluorescence-activated cell sorter (FACScan) (Becton and Dickenson, Heidelberg, Calif.) equipped with an argon ion laser (an excitation wavelength, 488 nM) and a doublet discrimination module (DDM). Lysis II (BD) software was used for data acquisition. Apoptotic nuclear changes were evaluated according to previously suggested criteria.

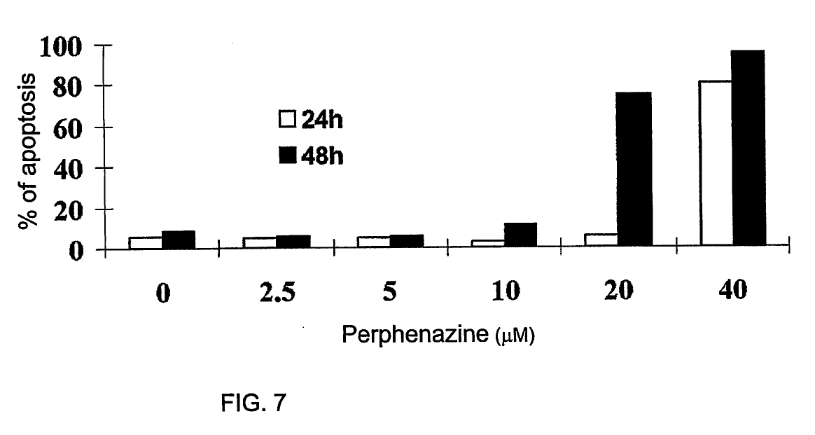

The results are shown in FIG. 7. As can be seen perphenazine induced a mortal and dramatic apoptosis after 24 and 48 hours at concentrations of 20 μM and higher.

### EXAMPLE 8

### Effect Psychotropic Drugs on p53 Mutant Gene Expression

The tumor suppressor protein p53 is a transcription factor involved in maintaining genomic integrity, and preventing cell proliferation. Mutations in the p53 gene are frequent in cancer diseases, and are associated with bad prognosis, development of resistance and difficulty to treatment.

The effects of the following agents: thioridazine, clozapine and perphenazine were assessed on the expression of p53 mutant gene in glioma C6 cell-line using western blotting analysis.

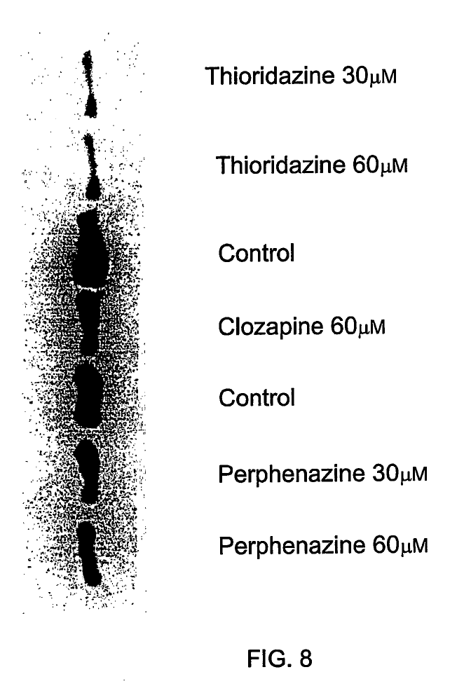

The data shown in FIG. 8 indicates a marked decrease in the expression of p53 mutant induced by the three agents, with highest activity of thioridazine (30 and 60 μM). (Mean 75% decrease), and mean of 30% by clozapine and perphenazine (60 μM).

### EXAMPLE 9

### Effects of Cyclic Antidepressants on Multi-Drug Resistant (MDR) Tumors

While some phenothiazines have been associated with some malignancies, there has been no explicit indications in the prior art that phenothiazines, or other cyclic antipsychotics are suitable in particular to tumors which were found to be resistant to other cytotoxic drugs.

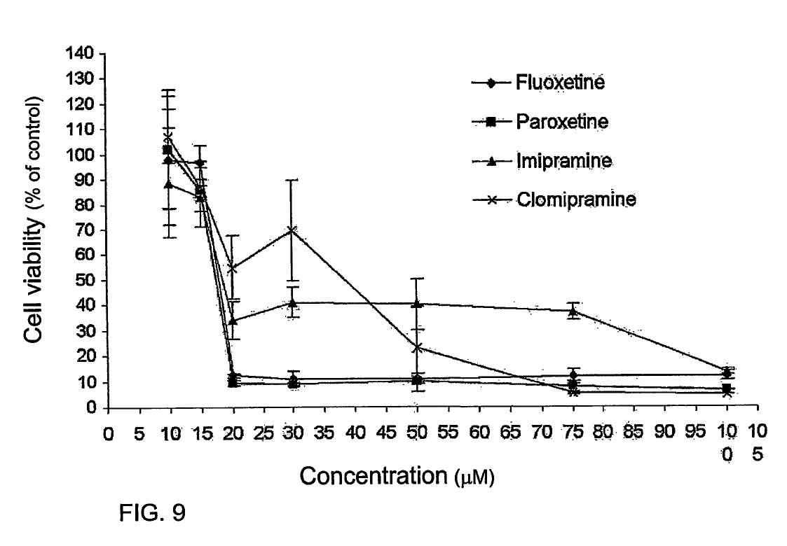

The effect of cyclic antidepressants, such as clomipramine, imipramine, fluoxetine and paroxetine on wild type B16 mouse melanoma and transformed (MDR) B16 melanoma cells was also tested. The results which are presented in FIG. 9 show a high sensitivity of both cell-lines to the cyclic antidepressant drugs, with IC50 levels of between 15-20 μM for paroxetine, fluoxetine, clomipramine and imipramine.

These results clearly indicate that cyclic antidepressants can inhibit survival of malignant cells resistant to doxorubicin.

### EXAMPLE 10

### In Vivo Studies of Thioridazine—Intra-Peritoneral Administration

### EXAMPLE 10(A)

Male C57 black mice aged 5-7 weeks were used. Animals were inoculated with B16 melanoma cells by iv. injection (200,000 cells/mouse) to the tail vein. Mice were treated with thioridazine (2.5, 5, 10 and 20 mg/kg i.p.×3 times/week), treatment was initiated one week before inoculation and continued after inoculation. Selection of concentration was performed after a preliminary experiment in which higher concentration of the drug (30, 50 and 100 mg/kg) were injected 3 times weekly and found toxic causing: sedation, respiratory depression and death. Body weight was recorded three times weekly during the experiment and survival was registered. Animals were sacrificed 24 days after cell inoculation, and lungs were dissected and weighted.

Results

The results (data not shown) teach that among treated animals survival rate was: for 2.5 mg/kg 4/5; for 5 mg/kg 3/4; for 10 and 20 mg/kg 5/5. Lungs weight was inversely related to the dose of thioridazine used, and a significant decrease in lungs weight was found in the group treated with 20 mg/kg compared to the low dose 2.5 mg/kg (p=0.05). No difference was found between the body weight pattern in the different groups.

### EXAMPLE 10(B)

Female C57 mice aged 5-7 weeks were used, animals were divided into 3 treatment groups:

1. Controls: B16 inoculated vehicle treated;

2. Thioridazine 10 mg/kg;

3. Thioridazine 15 mg/kg (injected i/p×3/week).

Half of the animals were sacrificed after 21 days and half after 27 days. Lungs weight and number of metastases were recorded.

Results

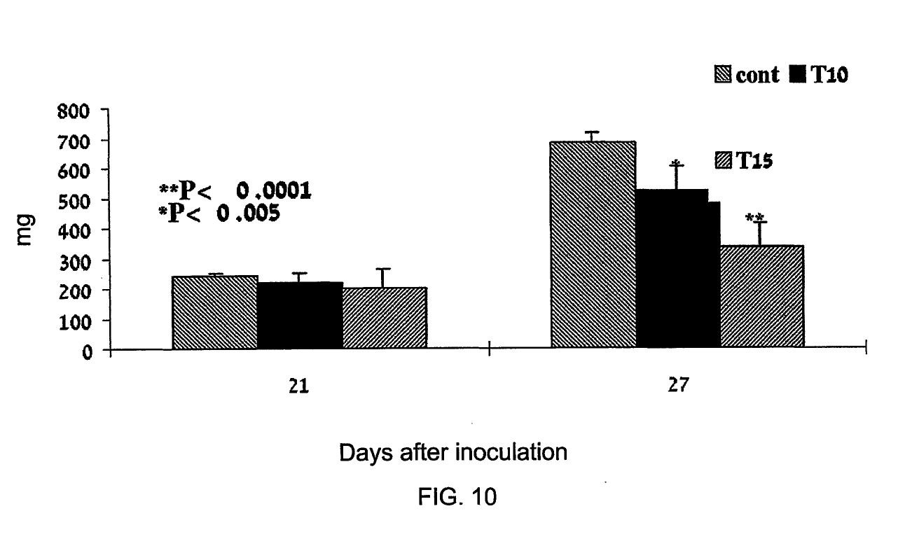

After 21 days animals showed a tendency toward decrease in metastases number in the thioridazine groups as compared to controls: mean number of metastases was: 28.5, 46.1 and 53.8+ for (Thio 15, Thio 10 mg/kg and controls respectively). No difference was found in lungs weight. When animals were sacrificed on the day 27 of the experiment, an inverse relationship was found between thioridazine dose and lungs weight. (Mean: 685, 520 and 335 mg for controls, Thio 10 mg/kg and Thio 15 mg/kg respectively). The difference between Thio 15 mg/kg and control animals was significant (p<0.05). In terms of number of metastases control animals presented confluent lungs in (6/7), Thio (10 mg/kg) showed also confluence in 6/7 mice, and Thio (15 mg/kg) showed confluence only in 2/7 mice. The results obtained (data not shown) are shown in FIG. 10 which shows that thioridazine administered via parenteral route seems to induce a parenteral activity in animals against tumor (B16 melanoma) growth and metastases spreading. The drug also increases survival rate. Effective doses were 15-20 mg/kg (lower doses were found not significantly effective). Concentrations higher than 30 mg/kg (parenteral route) were found toxic.

### EXAMPLE 11

### In Vivo Studies of Thioridazine—Administration Via Oral Route

### EXAMPLE 11(A)

Mice C57 black females ages 5-7 weeks were used. Mice were inoculated with Melanoma B16 cells and divided randomly (5-6/cage). Animals were divided into three groups:

1. Controls B16 with no therapy;

2. Thioridazine 20-30 mg/kg per day;

3. Thioridazine 30-40 mg/kg per day.

Thioridazine was dissolved in drinking water to form either a clear solution (i.e. at low concentration), or a very mild suspension (i.e. at high concentration). Water consumption was registered daily, and calculation of dose was performed according to the mean consumed water. Body weight was registered 3 time/week.

Results

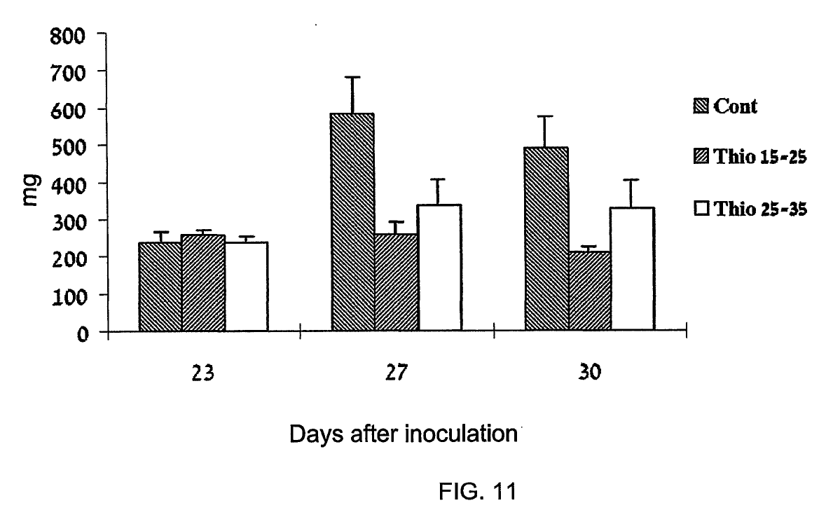

The drug was tolerated very well and no side effects or modification in behavior were noted in all animals. Animals were sacrificed after: 24, 27 and 30 days. After 24 days big or conglomerate metastases were only in 1/7 control mice, and the same rate was found in the 20-30 mg/kg treated group. No big or confluent metastases were found in the higher concentration group. In the next autopsies: 27 and 30 days were found a marked difference between the controls and the thioridazine treated groups: Mean lungs weight for controls B16 inoculated mice were: 583.4 and 487.6 mg, whereas mean lungs weight for thioridazine (Thio) groups were: 258.0, 209.4, 338.8 and 329.6 mg. Confluent metastases were found in 6/11 control animals and in 0/21 in Thio treated mice. Small size and low number of metastases were found also in Thio treated animals. Spontaneous death during experiment occurred in 5/18 controls and in 2/36 Thio treated mice. No difference was found in body weight between healthy controls, B16 controls and thioridazine treated mice. Results of lungs weight are shown in FIG. 11 and survival of mice given thioridazine (40-50 mg/kg) in drinking water results.

Oral administration of thioridazine (20-40 mg/kg/day) to mice inhibits tumor (B16 melanoma) growth, metastases spreading, and increases survival rate.

The drug (via oral administration) is well tolerated and no side effects were noted.

### EXAMPLE 12

### In Vivo Studies of Thioridazine in Combination with Doxorubicin—Administration Via Oral Route

Mice C57 black females ages 5-7 weeks were used. Mice were inoculated with Melanoma B16 cells and divided randomly (7-8/cage). Animals were divided into three groups:

1. Control-B16 cells with no therapy;

2. Treatment with doxorubicin 4 mg/kg i.p given once (3 days later).

3. Treatment with doxorubicin (4 mg/kg) and thioridazine 25-35 mg/kg/day via drinking water.

Results

All Animals in the control and the thioridazine+doxorubicin groups survived. In the doxorubicin group 2/7 animals died 7 and 14 days after inoculation. Animals were sacrificed after 21 days and lungs were dissected weighted and the number of metastases counted. Lungs weight was 770+/−95 mg in the B16 controls, 538+/−137 mg in the doxorubicin and 381+/−95 mg in the combined group. The number of animals presenting non confluent lungs was the highest 5/7 in the combined group compared to 2/7 in doxorubicin and 0/7 in the B16 control group.

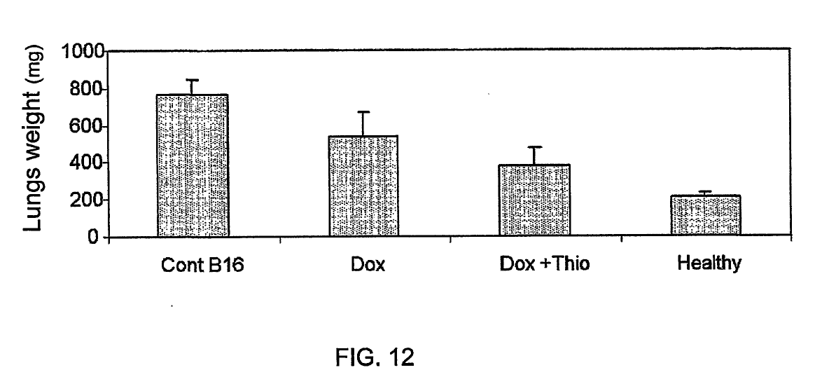

FIG. 12 and Table 2 below present, respectively, lungs weight and metastases status in the different mice.

TABLE 2
Effect on lung metastases of combined treatment with doxorubicin
and thioridazine

| Group | Total | Early death | Confluent | Non-confluent |
|---|---|---|---|---|
| 1 | 9 | 0 | 8 | 1 |
| 2 | 7 | 2 | 3 | 2 |
| 3 | 7 | 0 | 2 | 5 |

These results demonstrate that the combination of thioridazine and doxorubicin prevented spreading of metastases and improved the effect of doxorubicin on survival and tumor growth.

### EXAMPLE 13

### Effect of Psychotropic Drugs on Proliferative Skin Diseases

A. In Vitro Effect on Cell Viability

Materials and Methods

Three human immortal keratinocytes cell-lines were employed as models for proliferative disorders: HaCat (spontaneously immortalize, non tumorigenic human skin keratyniocyte line) HaCat IS (benign, tumorigenic), and HaCat II-4RT (Malignant Tumorigenic. The cells were maintained as described by Bachmeier BE et al. (Bachmeier BE et al. Biol. Chem 381(5-6):509-516 (2000)). Cells were treated with different classes of psychotropic drugs such as: phenothiazines (e.g. thioridazine, perphenazine), tricyclic neuroleptics (e.g. clozapine), tricyclic antidepressants (e.g. clomipramine, imipramine, doxepin), bicyclic antidepressants (e.g. paroxetine), monocyclic antidepressants (e.g. fluoxetine). The drugs were administered at concentrations within the range of 5-100 μM and cell viability was measured 24 hr post-administration by Neutral red staining. The efficiency of the agents against viability of the cell lines was evaluated also by comparison with two commonly used anticancer agents (Doxorubicin and 5-fluorouracil (5-FU)) at equimolar concentrations.

Results

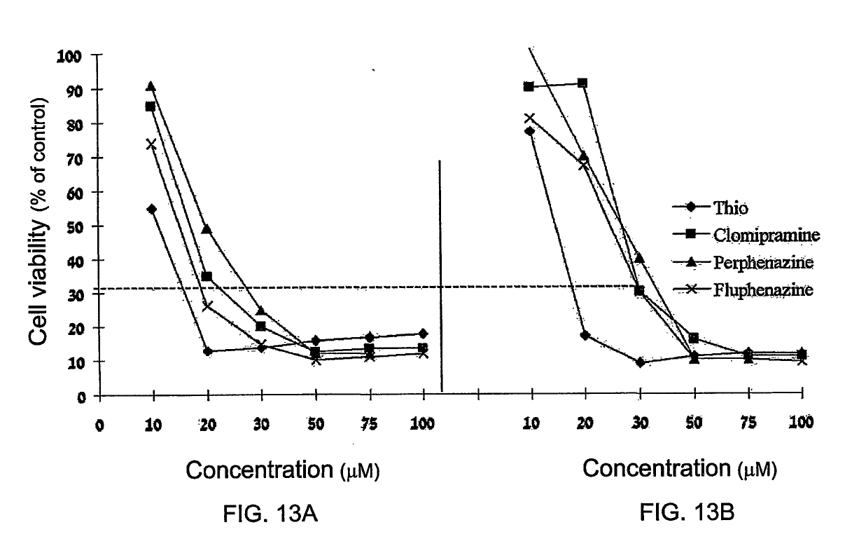

The different agents were shown to induce a marked dose dependent inhibitory activity on viability of the three different keratinocyte cell lines (FIGS. 13A and 13B). As presented in these figures thioridazine (Phenothiazines), clomipramine (tricyclic antidepressants) paroxetine (monocyclic antidepressant) and fluoxetine, (bicyclic antidepressant) were shown to be effective in reducing cell viability, i.e. inhibiting cell proliferation in both cell lines.

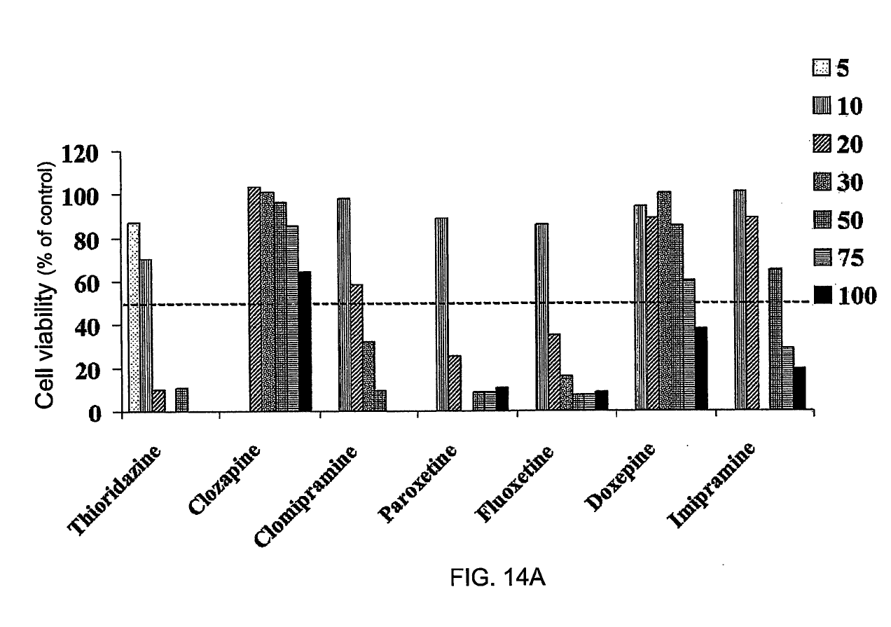

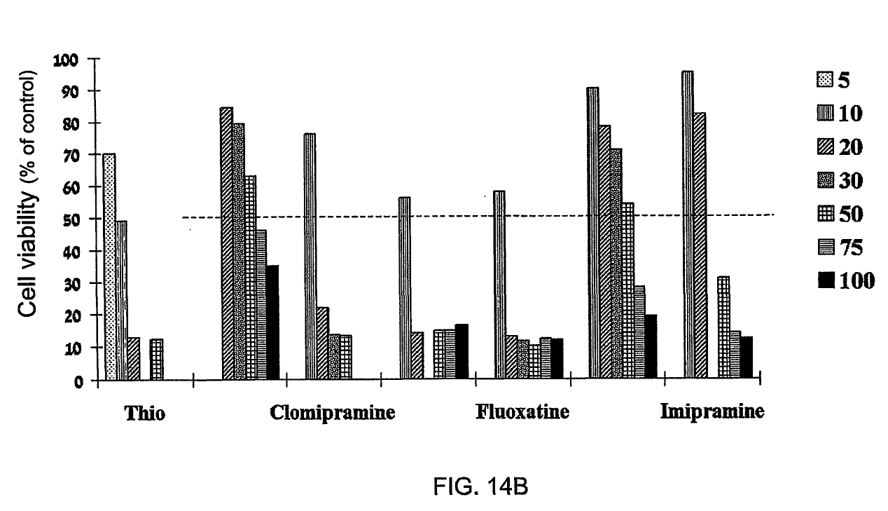

The sensitivity of the HaCat IIl-4RT malignant tumorigenic cell line was to the different agents was also evaluated and was shown to be higher than that of the non malignant (I5) cells or of the non tumorigenic HaCat cells, the latter showing a sensitivity similar to that of the I5 cell line. The IC50 values for the activity of the different agents on the three types of keratinocyte cell lines is summarized in Table 3 and FIG. 14A and FIG. 14B.

TABLE 3
IC50 (μM) in keratinocytes

| | HaCat | ME | HaCat I5 | ME | HaCat II4 |
|---|---|---|---|---|---|
| Thioridazine | 9, 14 | 11.5 | 13, 15, 15 | 14 | 10 |
| Perphenazine | 24, 24 | 24 | 30, 22 | 26 | |
| Fluphenazine | 16, 22 | 19 | 26, 18 | 22 | |
| Clomipramine | 20, 20 | 20 | 27, 31, 24 | 27 | 16 |
| Clozapine | >100 | >100 | >100 | >100 | 73 |
| Clotiapine | >100 | >100 | >100 | >100 | |
| Paroxetine | | | 21 | 21 | 12 |
| Fluoxetine | | | 20 | 20 | 13 |
| Doxepine | | | 62 | 62 | 48 |

As shown in Table 3, the IC50 values obtained for the active agents range between 9 μM and 100 μM, wherein the more active agents are considered as those possessing IC50 values of 10-30 μM.

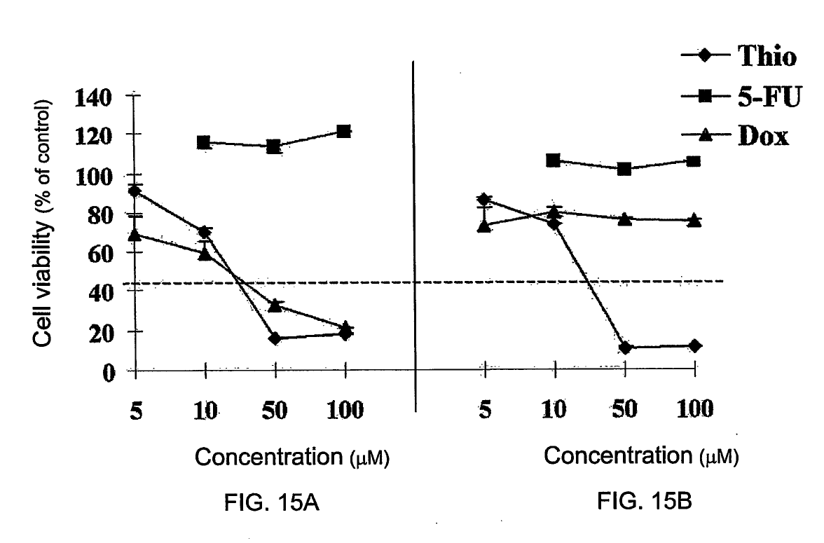

When responsiveness to doxorubicin and 5-FU as compared to thioridazine was tested in the HaCat and HaCat 15 cells (FIGS. 15A and 15B respectively), both cell-lines responded to thioridazine with a similar pattern of sensitivity, but were resistant to 5-FU, and only the HaCat (non tumorigenic) cells responded to doxorubicin with the same equimolar IC50 levels as for thioridazine.

B. Effect on DNA Fragmentation

DNA fragmentation was determined by flow cytometric analysis of propidium iodide-stained cells according to the method of Vindelov et al Vindelov, L. L., et al. Cytometry. 5:323-327 (1983)) using a fluorescence activated cell sorter (FACScan, Becton and Dickenson, Calif.). The study was conducted in HaCat and in HaCat 15 cells (500,000 and 1,000000 cells each sample) treated with 25 and 50 μM thioridazine.

HaCat cells show basal fragmentation rate of 29%, however, upon treatment with thioridazine the rate of fragmentation increased to a level of 82.8% (with 25 μM) and 89.3% (with 50 μM) respectively.

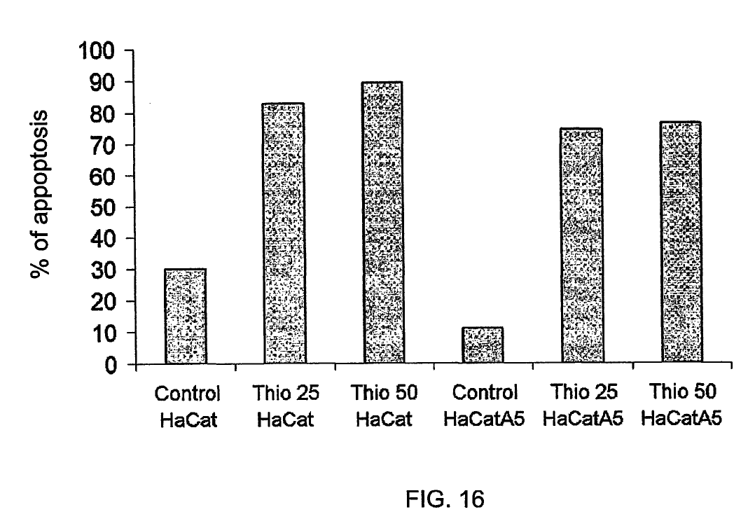

In I5 cell-line basal apoptosis was only 10.23% and following exposure to thioridazine, apoptosis increased to 74.5% (with 25 μM) and 76.6% (with 50 μM) respectively (see FIG. 16).

These results suggest that the inhibitory effect of thioridazine on the viability of proliferative skin cells is mediated by augmentation of DNA fragmentation, which is a hallmark of apoptotic mechanism.

C. Effect of Topical Administration of Thioridazine Cream on Psoriatic Subjects

Three subjects suffering from psoriasis, nevertheless, lacking any psychiatric disorder were treated for psoriasis with a cream containing thioridazine. Thioridazine cream was prepared by dissolving thioridazine (3 mg) in of distilled water (1.5 ml). The mixture was then added to a standard (aqueosum) eucerinum preparation (30 g) and mixed thoroughly until a homogenous cream was obtained.

Subject 1

An 18 years old female subject suffered since the age of 4 years from localized psoriasis with scaling and erythema mainly in elbows and knees (however, otherwise healthy and with no psychiatric disturbances or symptoms). The subject responded poorly to topical steroids. After several months without any treatment, the psoriatic areas of the subject's skin were applied twice a day with the cream.

A marked reducing in the skin's scaling and erythema were noticed even a few days after treatment, the improvement in the skin's condition persisted for one year during which the subject was continuously treated daily with the cream, as described above. In addition, the treatment was effective in reducing the size of the local lesions.

Upon cessation of the treatment (for 14 days) a marked exacerbation of the psoriatic lesions was observed which again were vanished after reestablishment of the treatment.

Subject 1

A 60 years old healthy male subject suffering from local psoriasis on the back and palms of his hands (however, otherwise healthy and with no psychiatric disturbances or symptoms) was treated twice daily with the thioridazine cream. After four months of treatment a decrease in the scales and erythema was observed. Cessation of treatment resulted in recurrence of the psoriatic symptoms.

With both subjects, no side effects of treatment were observed.

claims:

1. A method for the treatment of a proliferative disease comprising administering to a subject in need a therapeutically effective amount of at least one active ingredient, said active ingredient is a cyclic psychotropic agent selected from tricyclic neuroleptic and antipsychotics, bicyclic antidepressants and monocyclic antidepressants, with the proviso that said tricyclic neuroleptic and antipsychotic agents are not phenotiazines or thioxantenes and when said active ingredient is a monocyclic antidepressant, said proliferative disease is not prostate cancer.

2. The method of claim 1, wherein said tricyclic neuroleptic and antipsychotic agent is a derivative of dibenzothiepines, dibenzoazepines, dibenzothiazepines, dibenzodiazepines or dibenzooxazepines.

3. The method of claim 1 or 2, wherein said tricyclic neuroleptic and antipsychotic is clozapine or clotiapine.

4. The method of claim 1, wherein said bicyclic antidepressant is paroxetine.

5. The method of claim 1, wherein said monocyclic antidepressant is a phenylpropylamine derivative.

6. The method of claim 5, wherein said monocyclic antidepressant is a phenoxy-3-propylamine derivative selected from the group consisting of tomoxetine, nisoxetine and fluoxetine.

7. The method of claim 6, wherein said monocyclic antidepressant is fluoxetine.

8. The method of claim 1, wherein said proliferative disorder is cancer.

9. The method of claim 8, wherein said cancer is selected from neuroblastoma, glioma, melanoma, prostate cancer, multi-drug resistant (MDR) cancer, lung cancer, breast cancer and cancers associated with mutated p53 gene.

10. The method of claim 9, wherein said treatment comprises administration of said active ingredient in combination with a cytotoxic drug.

11. The method of claim 10, wherein said cytotoxic drug is doxorubicin.

12. The method of claim 1 comprising parenteral administration of the active ingredient.

13. The method of claim 12, wherein said parenteral administrations include intravenous, subcutaneous, intramuscular, intramedullary or direct injection.

14. The method of claim 1, comprising oral administration of the active ingredient.

15. A method for sensitizing proliferative cells to a cytotoxic drug comprising administering to a subject in need an amount of said cytotoxic drug in combination with a sensitizing amount at least one psychotropic agent with the proviso that said psychotropic agent is not a phenothiazine or a thioxantene.

16. The method of claim 15, wherein said psychotropic agent is a cyclic neuroleptic and antipsychotic agent or a cyclic antidepressant.

17. The method of claim 16, wherein said cyclic neuroleptic and antipsychotic agent is a tricyclic compound selected from clomipramine, amitriptyline, doxepin and imipramine.

18. The method of claim 17, wherein said cyclic antidepressant is a tricyclic compound selected from clozapine, and clotiapine.

19. The method of claim 16, wherein said cyclic antidepressant is paroxetine.

20. The method of claim 15, wherein said cytotoxic drug is doxorubicin.

21. A method for sensitizing MDR cancer cells to a cytotoxic drug comprising administering to a subject in need an amount of said cytotoxic drug in combination with a sensitizing amount of at least one psychotropic agent.

22. A method for sensitizing MDR cancer cells to a doxorubicin comprising administering to a subject in need an amount of doxorubicin in combination with a sensitizing amount of at least one psychotropic agent selected from clozapine, clomipramine, fluoxetine and paroxetine.

23. A method for sensitizing MDR cancer to a cytotoxic drug comprising administering to a subject in need an amount of said cytotoxic drug in combination with a sensitizing amount thioridazine.

24. The method of any one of claims 15, 21-23 comprising parenteral administration of the active ingredient.

25. The method according to claim 24, wherein said parenteral administrations include intravenous, subcutaneous, intramuscular, intramedullary or direct injection.

26. The method of any one of claims 15, 21-23, comprising oral administration of the active ingredient.

27. A method for the treatment of proliferative skin disorder which is not associated with psychiatric symptoms comprising administering to a subject in need a therapeutically effective amount of at least one psychotropic agent.

28. The method of claim 27, wherein said proliferative skin disorder is selected from psoriasis, hyperkeratosis and basal cell carcinoma.

29. The method of claim 27, wherein psychotropic agent is a phenothiazine.

30. The method of claim 29, wherein said phenothiazine is selected from the group consisting of thioridazine, perphenazine and fluphenazine.

31. The method of claim 27, wherein said psychotropic agent is a tricyclic antidepressant.

32. The method of claim 31, wherein said tricyclic antidepressant is selected from the group consisting of clomipramine, amitriptyline, doxepin and imipramine.

33. The method of claim 27, wherein said psychotropic agent is a bicyclic antidepressant.

34. The method of claim 33, wherein said bicyclic antidepressant is paroxetine.

35. The method of claim 26, wherein said psychotropic agent is a monocyclic antidepressant.

36. The method of claim 35, wherein said monocyclic antidepressant is fluoxetine.

37. The method of claim 27, wherein said active ingredient is applied topically to the diseases skin cells.

38. The method of claim 27, wherein said active ingredient is applied topically to the diseased skin cells.

39. A pharmaceutical composition for the treatment of proliferative diseases comprising a therapeutically effective amount of at least one active ingredient and a pharmaceutically acceptable carrier, said active ingredient is a cyclic psychotropic agent selected from tricyclic neuroleptic and antipsychotics, bicyclic antidepressants and monocyclic antidepressants, with the proviso that said tricyclic neuroleptic and antipsychotic agents are not phenotiazines or thioxantenes and when said active ingredient is a monocyclic antidepressant, said proliferative disease is not prostate cancer.

40. The composition of claim 39, wherein said tricyclic neuroleptic and antipsychotic agent is a derivative of dibenzothiepines, dibenzoazepines, dibenzothiazepines, dibenzodiazepines or dibenzooxazepines.

41. The composition of claim 39 or 40, wherein said tricyclic neuroleptic and antipsychotic is clozapine or clotiapine.

42. The composition of claim 39, wherein said bicyclic antidepressant is paroxetine.

43. The composition of claim 39, wherein said monocyclic antidepressant is a phenylpropylamine derivative.

44. The composition of claim 43, wherein said monocyclic antidepressant is a phenoxy-3-propylamine derivative selected from the group consisting of tomoxetine, nisoxetine and fluoxetine.

45. The composition of claim 44, wherein said monocyclic antidepressant is fluoxetine.

46. The composition of claim 39, wherein said proliferative disorder is cancer.

47. The composition of claim 46, wherein said cancer is selected from neuroblastoma, glioma, melanoma, prostate cancer, multi-drug resistant (MDR) cancer, lung cancer, breast cancer and cancers associated with mutated p53 gene.

48. The composition of claim 39, comprising a cytotoxic drug.

49. The composition of claim 48, wherein said cytotoxic drug is doxorubicin.

50. The composition according to claim 39, in a dosage form suitable for parenteral administration.

51. The composition according to claim 50, in a dosage suitable for intravenous, subcutaneous, intramuscular, intramedullary or direct injection.

52. The composition of claim 39, in a dosage form suitable for oral administration.

53. A pharmaceutical composition for sensitizing proliferative cells to a cytotoxic drug comprising an amount of said cytotoxic drug, a sensitizing effective amount of a psychotropic agent and a pharmaceutically acceptable carrier with the proviso that said psychotropic agent is not a phenothiazine or a thioxantene.

54. The composition of claim 53, wherein said psychotropic agent is a cyclic neuroleptic and antipsychotic agent or a cyclic antidepressant agent.

55. The composition of claim 54, wherein said cyclic neuroleptic and antipsychotic agent is a tricyclic compound selected from clomipramine, amitriptyline, doxepin and imipramine.

56. The composition of claim 55, wherein said cyclic agent is a tricyclic compound is selected from clozapine and clotiapine.

57. The composition of claim 53, wherein said cyclic antidepressant is paroxetine.

58. The composition of claim 53, wherein said cytotoxic drug is doxorubicin.

59. A pharmaceutical composition for sensitizing MDR cancer cells to a cytotoxic drug comprising an amount of said cytotoxic drug in combination with a sensitizing amount of at least one psychotropic agent.

60. A pharmaceutical composition for sensitizing MDR cancer cells to doxorubicin comprising an amount of doxorubicin and a sensitizing amount of at least one psychotropic agent selected from clozapine, clomipramine, fluoxetine and paroxetine.

61. A pharmaceutical composition for sensitizing MDR cancer cells to a cytotoxic drug comprising an amount of said cytotoxic drug, a sensitizing effective amount thioridazine and a pharmaceutically acceptable carrier.

62. The composition of any one of claims 53 or 60-61, wherein said pharmaceutically acceptable carrier is suitable for parenteral administration.

63. The composition according to claim 62, in a dosage form suitable for intravenous, subcutaneous, intramuscular, intramedullary or direct injection.

64. The composition of any one of claims 53 or 60-61, in a dosage form suitable for oral administration.

65. A pharmaceutical composition for the treatment of proliferative skin disorders which are not associated with psychiatric symptoms comprising a therapeutically effective amount of a psychotropic agent and a pharmaceutically acceptable carrier.

66. The composition of claim 65, wherein said proliferative skin disorder is selected from psoriasis, hyperkeratosis and basal cell carcinoma.

67. The composition of claim 65, wherein psychotropic agent is a phenothiazine.

68. The composition of claim 67, wherein said phenothiazine is selected from the group consisting of thioridazine, perphenazine and fluphenazine.

69. The composition of claim 65, wherein said psychotropic agent is a tricyclic antidepressant.

70. The composition of claim 69, wherein said tricyclic antidepressant is selected from the group consisting of clomipramine amitriptyline, doxepin and imipramine.

71. The composition of claim 65, wherein said psychotropic agent is a bicyclic antidepressant.

72. The composition of claim 71, wherein said bicyclic antidepressant is paroxetine.

73. The composition of claim 65, wherein said psychotropic agent is a monocyclic antidepressant.

74. The composition of claim 73, wherein said monocyclic antidepressant is fluoxetine.

75. The composition of claim 65, wherein said pharmaceutically acceptable carrier is suitable for application of the composition topically onto the hyper-proliferating skin.

76. Use of a cyclic psychotropic agent selected from tricyclic neuroleptic and antipsychotics, bicyclic antidepressants and monocyclic antidepressants for the preparation of a pharmaceutical composition for the treatment of proliferative diseases, with the proviso that said tricyclic neuroleptic and antipsychotic agents are not phenotiazines or thioxantenes and that when said active ingredient is a monocyclic antidepressant, said proliferative disease is not prostate cancer.

77. The use of claim 76, wherein said tricyclic neuroleptic and antipsychotic agent is a derivative of dibenzothiepines, dibenzoazepines, dibenzothiazepines, dibenzodiazepines or dibenzooxazepines.

78. The use of claim 76 or 77, wherein said tricyclic neuroleptic and antipsychotic is clozapine or clotiapine.

79. The use of claim 76, wherein said bicyclic antidepressant is paroxetine.

80. The use of claim 76, wherein said monocyclic antidepressant agent is a phenylpropylamine derivative.

81. The use of claim 80, wherein said monocyclic antidepressant agent is a phenoxy-3-propylamine derivative selected from the group consisting of tomoxetine, nisoxetine and fluoxetine.

82. The use of claim 81, wherein said monocyclic antidepressant agent is fluoxetine.

83. The use of claim 76, for the preparation of a pharmaceutical composition for the treatment of cancer.

84. The use of claim 83, wherein said cancer is selected from neuroblastoma, glioma, melanoma, prostate cancer, multi-drug resistant (MDR) cancer, lung cancer, breast cancer and cancers associated with mutated p53 gene.

85. Use of a psychotropic agent for the preparation of a pharmaceutical composition for sensitizing proliferative cells to a cytotoxic drug, with the proviso that said psychotropic agent is not a phenothiazine or a thioxantene

86. The use of claim 85, wherein said psychotropic agent is a cyclic neuroleptic and antipsychotic agent or a cyclic antidepressant agent.

87. The use of claim 86, wherein said cyclic neuroleptic and antipsychotic agent is a tricyclic compound.

88. The use of claim 87, wherein said tricyclic compound is clozapine, clomipramine.

89. The use of claim 86, wherein said cyclic antidepressant is paroxetine.

90. The use of claim 85, wherein said cytotoxic drug is doxorubicin.

91. Use of a psychotropic agent for the preparation of a pharmaceutical composition for sensitizing MDR cancer cells to a cytotoxic drug.

92. The use of a psychotropic agent for the preparation of pharmaceutical composition for sensitizing MDR cancer cells to doxorubicin, wherein said psychotropic agent is selected from clozapine, clomipramine, fluoxetine and paroxetine.

93. Use of thioridazine for the preparation of a pharmaceutical composition for sensitizing MDR cancer cells to a cytotoxic drug.

94. Use of a psychotropic agent for the preparation of a pharmaceutical composition for the treatment proliferative skin disorders which are not associated with psychiatric symptoms.

95. The use of 94, wherein said proliferative skin disorder is selected from psoriasis, hyperkeratosis and basal cell carcinoma.

96. The use of claim 94, wherein said psychotropic agent is a phenothiazine.

97. The use of claim 96, wherein said phenothiazine is selected from the group consisting of thioridazine, perphenazine and fluphenazine.

98. The use of claim 94, wherein said psychotropic agent is a tricyclic antidepressant.

99. The use of claim 98, wherein said tricyclic antidepressant is selected from the group consisting of clomipramine amitriptyline, doxepin and imipramine.

100. The use of claim 99, wherein said psychotropic agent is a bicyclic antidepressant.

101. The use of claim 100, wherein said bicyclic antidepressant is paroxetine.

102. The use of claim 101, wherein said psychotropic agent is a monocyclic antidepressant.

103. The use of claim 102, wherein said monocyclic antidepressant is fluoxetine.
